(19) United States
(12) Patent Application Publication
(10) Pub. No.: US 2026/0294866 A1
(43) Pub. Date: Oct. 1, 2026

(54) Multi Ingredient Natural Products Composition And Method Of Use

(71) Applicant: Visionary Ventures LLC, Spearfish, SD (US)

(72) Inventors: Mark R. Gordon, Spearfish, SD (US); Veronica Davis-Gordon, Vista, CA (US)

(73) Assignee: Visionary Ventures LLC, Vista, CA (US)

(21) Appl. No.: 19/096,231

(22) Filed: Mar. 31, 2025

(51) Int. Cl. A61K 31/37; A61K 31/132

(52) U.S. Cl. CPC A61K 31/37; A61K 31/132

(57) ABSTRACT

A multi-ingredient natural products composition for promoting mitochondrial health and autophagy includes a formulation that includes urolithin A and spermidine, or a biologically active analog thereof. The formulation also includes one or more of a liquid, an excipient, and a device that render the formulation suitable for therapeutic delivery to a subject to improve a biological process in the subject leading to enhanced longevity and cellular health for the subject.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to natural products compositions and more particularly pertains to a new natural products composition for promoting mitochondrial health and autophagy. While a variety of natural products compositions are known in the prior art, there remains a need for novel natural products compositions that are specifically directed to the enhancement of longevity and cellular health. Therefore, a natural products composition containing urolithin A and spermidine in a formulation that is readily bioavailable would be beneficial in supporting mitochondrial function, autophagy, and overall cellular health.

### (2) Description of Related Art Including Information Disclosed Under 37 CFR 1.97 and 1.98

The prior art relates to natural products compositions, but which do not comprise urolithin A and spermidine in a readily bioavailable format. While many existing interventions for age-related decline are known in the prior art, they concentrate on isolated mechanisms and fail to provide a comprehensive strategy to promote mitochondrial health and autophagy, resulting in incomplete alleviation of cellular dysfunction, diminished energy production, and progressive tissue damage. Current therapeutic avenues often neglect the selective removal of damaged mitochondria, leading to suboptimal regeneration of healthy organelles and persistent metabolic inefficiencies that exacerbate oxidative stress, reduce muscle performance, and hasten overall functional deterioration. Formulations aimed at enhancing autophagy and mitigating inflammatory processes generally suffer from insufficient bioavailability or inadequate synergistic action, preventing the effective, simultaneous targeting of multiple pathways such as cardiovascular function, cognitive vitality, and cellular cleanup processes, thereby limiting their capacity to produce significant longevity and health span improvements.

The endogenous production of urolithin A and spermidine is limited in humans. Urolithin A is synthesized from ellagitannins through gut microbial metabolism; however, only a subset of the population possesses the specific gut microbiota required for this conversion. Studies indicate that approximately 10% of individuals are capable of producing urolithin A from dietary precursors. Similarly, while spermidine is naturally present in the body, its levels decline with age and dietary intake may not compensate sufficiently for this reduction.

### BRIEF SUMMARY OF THE INVENTION

An embodiment of the disclosure meets the needs presented above by generally comprising a formulation, which in turn comprises urolithin A and spermidine and one or more of a liquid, an excipient, and a device configured to render the formulation suitable for therapeutic delivery to a subject. The formulation thus is configured to improve a biological process in the subject leading to enhanced mitochondrial function, autophagy, and overall cellular health for the subject. The urolithin A and the spermidine are present in specific concentrations to synergistically activate pathways such as Nrf2, AMPK, sirtuins, and mTOR inhibition. The formulation reduces oxidative stress, inflammation, and cellular damage while enhancing vascular function, cognitive health, energy metabolism, and cellular renewal. The formulation supports hair growth, enhances clear skin and complexion, and creates hormone balance in men and women. The formulation provides a comprehensive solution for improving longevity, mitigating age-related decline, and promoting overall physiological health.

Another embodiment of the disclosure includes a method of treating a subject with the formulation to improve a biological process in the subject. The method entails provision of the urolithin A, the spermidine, and the one or more of the liquid, the excipient, and the device, as per the disclosure above. Upon producing the formulation, it can be delivered to the subject to improve the biological process.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a block diagram of a multi-ingredient natural products composition according to an embodiment of the disclosure.

FIG. 2 is a flowchart for a method utilizing an embodiment of the disclosure.

### DETAILED DESCRIPTION OF THE INVENTION

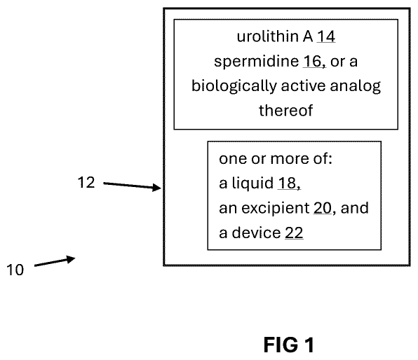

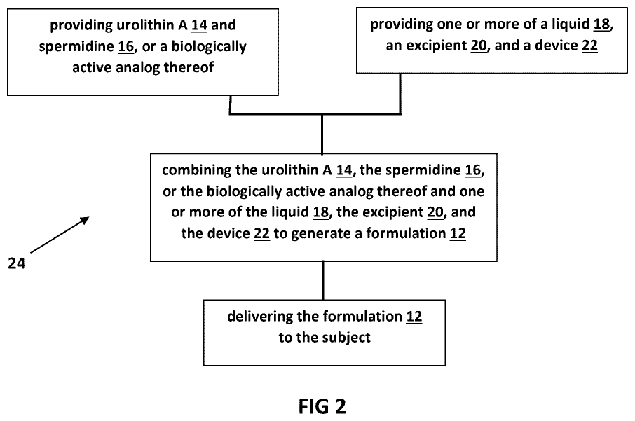

With reference now to the drawings, and in particular to FIGS. 1 and 2 thereof, a new natural products composition embodying the principles and concepts of an embodiment of the disclosure and generally designated by the reference numeral 10 will be described.

As best illustrated in FIGS. 1 and 2, the multi-ingredient natural products composition 10 generally comprises a formulation 12 comprising urolithin A 14, spermidine 16, and one or more of a liquid 18, an excipient 20, and a device 22 configured to render the formulation 12 suitable for therapeutic delivery to a subject. “Spermidine,” in the context of this disclosure, should be interpreted to mean the spermidine 16 or any biologically active analog thereof. The formulation 12 thus is configured to improve a biological process in the subject leading to enhanced longevity and cellular health for the subject.

The one or more of the liquid 18, the excipient 20, and the device 22 of the multi-ingredient natural products composition 10 may be any liquid 18, excipient 20, or device 22 that facilitates the transport, release, and subsequent administration of the urolithin A 14 and the spermidine 16 to the subject, encompassing various routes such as oral, parenteral, transdermal, inhalational, or otherwise, wherein the design parameters, including dosage form, release profile, and composition of excipients 20 are selected to optimize bioavailability, stability, subject compliance, and therapeutic efficacy.

For example, the formulation 12 may be rendered as a pill, a tablet, a capsule, or other solid oral dosage form comprising one or more excipients 20 such as, but not limited to, binding agents, disintegrants, inert or minimally active fillers, or the like, which can be compressed, encapsulated, or the like, to produce a stable unit that is convenient for oral administration. Inert or minimally active fillers include, but are not limited to, lactose, cellulose derivatives, starch, calcium phosphate salts, various sugars, various sugar alcohols, and numerous other pharmaceutically acceptable inert or minimally active fillers that may be used depending on the dosage form. Inert or minimally active fillers are selected based on their ability to ensure content uniformity, to support tablet binding or flow properties, and to maintain chemical stability. By modulating compressibility, dissolution rates, or bioavailability, the inert or minimally active fillers help produce a consistently dosed and stable formulation 12 that adheres to stringent regulatory, safety, and efficacy requirements.

In some embodiments, the formulation 12 is rendered as a time-release capsule or transdermal patch to ensure sustained bioavailability of the urolithin A 14 and the spermidine 16. In other embodiments, the formulation 12 may be rendered as a solution, suspension, or the like, which may require dissolving the urolithin A 14 and spermidine 16 in a liquid 18 that is a pharmaceutically acceptable solvent system or uniformly dispersing the urolithin A 14 and spermidine 16 in a liquid 18 that acts as a suspension medium, potentially with surfactants, viscosity-modifying polymers, or stabilizing excipients 20. The resulting formulation 12 can be administered orally, parenterally, or via other routes as deemed appropriate.

The excipient 20 may comprise a flavoring agent, which is incorporated into the formulation 12 to enhance flavor or to mask inherent flavor, thereby improving palatability and subject compliance. The flavoring agent may be derived from natural sources, such as essential oils and fruit extracts, or they may be synthesized chemically. The flavoring agent is selected based on their stability during manufacturing and throughout the product's shelf life. The flavoring agent also can include sweeteners, specialized taste-masking agents, and synthetic or natural flavor compounds, all of which are designed to impart or modify sensory attributes such as sweetness, bitterness, or acidity while remaining compatible with the urolithin A 14 and spermidine 16 and other excipients 20.

The excipient 20 may comprise a preservative, which may have antimicrobial or fungistatic properties. The preservative is incorporated into the formulation 12 to inhibit microbial growth and maintain integrity of the formulation 12 throughout its shelf life. Common examples of the preservative include, but are not limited to, parabens, benzoic acid, and sorbic acid, along with their corresponding salts. Additionally, phenolic compounds that effectively reduce or eliminate bacterial, fungal, or other microbial contamination can serve as the preservative. The preservative may be selected based on its spectrum of activity, stability within the formulation 12, and regulatory acceptance in accordance with established safety and efficacy guidelines. Formulators can optimize the concentration and compatibility of the preservative to ensure that it does not adversely affect the urolithin A 14 and the spermidine 16 or the taste or the physicochemical properties of the formulation 12 while preserving overall quality and subject acceptability properties of the formulation 12.

The urolithin A 14 of the formulation 12 is a bioactive postbiotic compound structurally classified as a benzocoumarin derivative known by the chemical name 3,8-Dihydroxy-6H-dibenzo[b,d]pyran-6-one. The urolithin A 14 may be formed through the intestinal microbial conversion of ellagitannins and ellagic acid, which are naturally present in pomegranate, certain berry species, and various nuts. In commercial production or research settings, the urolithin A 14 may be obtained via precision fermentation techniques or synthesized through multi-step organic chemistry processes that isolate and purify the target molecule to high standards of quality and consistency. The urolithin A 14 may have potential longevity-enhancing effects, primarily through upregulation of mitophagy, a quality-control mechanism wherein senescent or damaged mitochondria are selectively targeted and removed, ensuring enhanced mitochondrial function and energy metabolism. By promoting this targeted turnover of dysfunctional organelles, the urolithin A 14 may reduce cellular oxidative stress, restore homeostatic processes, and improve the overall bioenergetic capacity of cells. Furthermore, emerging research suggests that the urolithin A 14 may have beneficial interactions with molecular pathways involved in aging, such as those regulated by AMP-activated protein kinase (AMPK) or sirtuins, adding another layer of potential synergy in bolstering cellular resilience, delaying age-related decline, and improving overall physiological function. The urolithin A 14 may be effective at a dose as low as 200 mg, although the formulation 12 may include up to 1500 mg of the urolithin A 14.

The spermidine 16 of the formulation 12 is naturally occurring polyamine known by the chemical name N1-(3-Aminopropyl)butane-1,4-diamine. The spermidine 16 may be integral to cellular homeostasis, where it regulates cell growth, proliferation, and differentiation by modulating essential biological processes such as DNA stabilization, RNA transcription, and protein synthesis. In both eukaryotic and prokaryotic systems, the spermidine 16 biosynthesis involves the enzymatic decarboxylation of S-adenosylmethionine to form the precursor decarboxylated S-adenosylmethionine, which subsequently transfers an aminopropyl moiety to putrescine, yielding spermidine 16. Industrial production of the spermidine 16 frequently employs microbial fermentation routes in which strains of bacteria or yeast are genetically engineered or selectively cultivated to accumulate elevated levels of polyamines. Cell biomass then is harvested and subjected to downstream processing, typically involving cell lysis, filtration, and chromatographic steps, to obtain the spermidine 16 at a purity suitable for dietary or therapeutic use. In the context of longevity, the spermidine 16 may have the capacity to induce autophagy, a conserved catabolic process that sequesters, degrades, and recycles damaged organelles, misfolded proteins, and other intracellular debris. By promoting efficient autophagic turnover, the spermidine 16 may attenuate the accumulation of cellular damage and oxidative stress that often coincide with aging, thereby favorably impacting mitochondrial function, metabolic regulation, and inflammatory status. Research in model organisms has shown that supplementation with exogenous spermidine 16 may extend the median and maximum lifespan by enhancing stress resistance and preserving tissue integrity. Although further clinical validation is warranted, these mechanistic and preclinical findings collectively indicate that the spermidine 16 may exert health span-promoting and potentially life-extending effects through multifaceted pathways involving autophagy, immune modulation, and improved proteostasis. The spermidine 16 may be effective at a dose as low as 0.5 mg, although the formulation 12 may include up to 20.0 mg of the spermidine 16.

The urolithin A 14 and the spermidine 16 may be blended with the fillers, the flavoring agent, and the preservative in accordance with the procedures described above. Typical dosage ranges for urolithin A 14 and spermidine 16 within the formulation 12 are from 200 mg to 1500 mg for the urolithin A 14 and 0.5 mg to 20.0 mg for the spermidine 16. Even more typically, the dosage ranges for the urolithin A 14 and the spermidine 16 within the formulation 12 are from 250 mg to 1000 mg for the urolithin A 14 1.0 mg to 10.0 mg for the spermidine 16. The present invention anticipates the urolithin A 14 and the spermidine 16 being present in the formulation 12 in optimized ratios designed to maximize the activation of mitophagy and autophagy while maintaining optimal bioavailability. The ratio of the urolithin A 14 to the spermidine 16 in the formulation 12 typically will have a range of from 25:1 to 1000:1 by weight and the formulation 12 delivers effective therapeutic doses without pharmacokinetic interference.

Addition of complementary agents (e.g., vitamins, minerals, or other phytochemicals) may be utilized to create alternative formulations 12 consistent with the present disclosure's objective of modulating key longevity pathways, thereby allowing broad latitude for tailoring precise nutritional or therapeutic interventions to enhance mitochondrial function, autophagy, and overall cellular health for the subject.

Table 1 provides putative functional activities and highlights the additive and synergistic benefits of the urolithin A 14 and the spermidine 16. The rows in Table 1 correspond to the urolithin A 14 and the spermidine 16 alone and in combination. The columns of Table 1 outline key functional benefits. A detailed discussion of these functional activities and their role in enhancing longevity and cellular health for the subject follows Table 1.

TABLE 1
Putative Functional Activities of Urolithin A 14, Spermidine 16, and a Combination Thereof

| Natural | Mitochondrial | Autophagy | Inflammation | Oxidative Stress | Cognitive |
|---|---|---|---|---|---|
| Product(s) | Function | Induction | Reduction | Mitigation | Support |
| Urolithin A 14 | Promotes | N/A | Reduces | Activates | Enhances |
| | mitophagy, | | oxidative stress | AMPK, | neuroprotection |
| | improving | | by removing | reduces | through |
| | mitochondrial | | dysfunctional | ROS | mitochondrial |
| | efficiency | | mitochondria | generation | renewal |
| Spermidine 16 | Enhances | Induces | Suppresses | Reduces | Supports |
| | mitochondrial | autophagy, | inflammatory | cellular | proteostasis for |
| | biogenesis via | removing | cytokines | oxidative | improved cognitive |
| | autophagy | intracellular | | stress | function |
| | | debris | | | |
| Combination | Synergistically | Amplifies | Suppresses | Reduces | Preserves cognitive |
| | enhances | autophagic | inflammation | systemic | performance by |
| | mitochondrial | turnover | through | oxidative | protecting neuronal |
| | energy | | combined | stress | cells |
| | production | | pathway | | |
| | | | modulation | | |

The synergistic interplay between the urolithin A 14 and the spermidine 16 mitochondrial quality control through the simultaneous activation of mitophagy and autophagy. The urolithin A 14 promotes the targeted removal of senescent or dysfunctional mitochondria, thereby improving mitochondrial efficiency and reducing oxidative stress, while the spermidine 16 facilitates the clearance of intracellular debris, including misfolded proteins and damaged organelles. Together, these mechanisms act to amplify cellular rejuvenation, enhance bioenergetic capacity, and maintain physiological homeostasis, producing benefits that exceed the additive effects of either the urolithin A 14 or the spermidine 16 administered individually.

The formulation 12 uniquely influences key signaling pathways associated with aging and cellular health, including mTOR, AMPK, and NAD⁺ metabolism. The urolithin A 14 and the spermidine 16 synergistically modulate mTOR signaling to promote cellular stress resistance and to delay age-related decline. The urolithin A 14 upregulates AMPK and sirtuin activity, while the spermidine 16 enhances NAD⁺ levels indirectly through autophagy-driven metabolic regulation, collectively promoting mitochondrial biogenesis and improving cellular resilience. The combinatorial action ensures comprehensive coverage of age-related pathways by targeting proteostasis, immune modulation, and oxidative stress mitigation for improved health span and longevity.

The formulation 12 extends its utility by addressing secondary health benefits derived from the synergistic action of its components. The enhanced mitochondrial function achieved through administration of the urolithin A 14 and the spermidine 16 in the formulation 12 supports cognitive performance and neuroprotection, making the composition suitable for addressing age-related cognitive decline. Additionally, the reduction of systemic inflammation and oxidative stress contributes to improved vascular health, metabolic efficiency, and tissue regeneration. The formulation 12 is further effective in specific subject populations, including elderly individuals seeking to preserve muscle mass and endurance, or patients managing chronic conditions associated with mitochondrial dysfunction.

As shown in Table 1, the urolithin A 14 primarily promotes mitochondrial function by inducing mitophagy, a selective quality control mechanism that removes senescent or damaged mitochondria. This process reduces oxidative stress, restores cellular bioenergetics, and enhances overall mitochondrial efficiency. In contrast, the spermidine 16 induces autophagy, facilitating the clearance of intracellular debris, such as misfolded proteins and damaged organelles. By targeting complementary pathways, the formulation 12 achieves superior outcomes in cellular cleanup, energy production, and inflammation reduction.

The formulation 12 amplifies autophagic turnover and mitigates systemic oxidative stress. This synergistic effect of the urolithin A 14 and the spermidine 16 is particularly evident in pathways that regulate mitochondrial renewal and energy metabolism, such as AMPK activation. For example, the urolithin A 14 activates mitophagy to remove dysfunctional mitochondria, while the spermidine 16 simultaneously enhances mitochondrial biogenesis. Together, these mechanisms improve mitochondrial density, energy efficiency, and resistance to oxidative damage.

The formulation 12 addresses multiple hallmarks of aging through distinct biochemical pathways. The urolithin A 14 enhances mitophagy, facilitating the removal of damaged mitochondria, thereby mitigating mitochondrial dysfunction and reducing oxidative stress. The spermidine 16 induces autophagy, promoting the clearance of misfolded proteins and maintaining proteostasis. Additionally, the spermidine 16 modulates epigenetic regulation and thereby influences gene expression patterns associated with aging. Both the urolithin A 14 and the spermidine 16 exhibit anti-inflammatory properties to address chronic inflammation and support genomic stability by reducing DNA damage. Collectively, these actions contribute to improved cellular function and longevity.

Additionally, the formulation 12 reduces inflammation through suppression of key inflammatory cytokines and modulation of signaling pathways such as mTOR and sirtuins. The urolithin A 14 reduces oxidative stress by removing damaged mitochondria, while the spermidine 16 indirectly suppresses inflammatory markers by enhancing autophagic turnover. This dual action supports cardiovascular and metabolic health by reducing chronic low-grade inflammation commonly associated with aging.

Cognitive support is achieved through the neuroprotective effects of the formulation 12 disclosed herein. The urolithin A 14 enhances mitochondrial renewal in neuronal cells, providing improved energy production and reduced oxidative damage. The spermidine 16 supports proteostasis, reducing the accumulation of damaged proteins and cellular debris that impair cognitive function. Together, these effects preserve neuronal integrity and enhance cognitive resilience in aging individuals.

The synergistic effects of the urolithin A 14 and the spermidine 16 in the formulation 12 demonstrates an ability to target and enhance interconnected biochemical pathways associated with aging and cellular health. By working through complementary mechanisms, the urolithin A 14 and the spermidine 16 amplify each other's beneficial effects, resulting in a formulation 12 that significantly extends health span and improves physiological resilience.

The urolithin A 14 enhances mitochondrial quality control by stimulating mitophagy, a selective process for clearing damaged mitochondria. This function restores mitochondrial efficiency, reduces oxidative stress, and enhances bioenergetics. Simultaneously, the spermidine 16 induces autophagy, facilitating the removal of damaged cellular components, including misfolded proteins and other intracellular debris. Together, these mechanisms synergistically improve cellular cleanup, ensuring both mitochondrial and cytoplasmic health.

Both the urolithin A 14 and the spermidine 16 independently influence key metabolic pathways, including AMPK activation and mTOR inhibition. The urolithin A 14 activates AMPK, enhancing fatty acid oxidation and insulin sensitivity while also inhibiting the mTOR pathway to promote autophagic activity and cellular repair. The spermidine 16 complements this by further inhibiting mTOR signaling and activating SIRT1, a longevity-associated protein that regulates stress resistance and metabolic health. This dual targeting of AMPK and mTOR ensures comprehensive modulation of energy metabolism, contributing to improved weight management, metabolic regulation, and insulin sensitivity.

The formulation 12 also addresses oxidative stress through enhanced activation of the Nrf2 pathway. The urolithin A 14 and the spermidine 16 each upregulate antioxidant defenses by increasing the expression of antioxidant enzymes such as superoxide dismutase (SOD) and catalase. This collaborative action reduces cellular damage caused by reactive oxygen species (ROS) and protects against age-related oxidative damage, contributing to improved cellular longevity and systemic health.

In addition to these cellular benefits, the formulation 12 demonstrates profound anti-inflammatory effects. The urolithin A 14 modulates inflammatory responses by targeting cytokines and reducing oxidative stress, while the spermidine 16 suppresses inflammatory markers through its autophagy-enhancing effects. Together, the urolithin A 14 and the spermidine 16 synergistically reduce chronic low-grade inflammation, a hallmark of aging and age-related diseases.

The neuroprotective effects achieved by the formulation 12 further highlight its potential. By enhancing mitophagy and autophagy in neuronal cells, formulation 12 preserves neuronal integrity, reduce neuroinflammation, and improve cognitive resilience. These neuroprotective effects are supported by the reduction of oxidative stress and inflammation, as well as improved mitochondrial efficiency, which collectively protect against neurodegenerative diseases and support cognitive performance in aging populations.

Moreover, the actions of formulation 12 extend beyond cellular health to address systemic physiological functions. Improved mitochondrial function and autophagy contribute to enhanced muscle health and endurance, reduced cardiovascular risks, and better metabolic health. The effects of the formulation 12 on tissue regeneration and repair make it suitable for individuals managing chronic conditions or seeking to preserve physical vitality as they age.

In some embodiments, the formulation 12 may optionally include other ingredients with therapeutic benefits to further enhance their synergistic effects. These additional compounds may include adaptogenic ingredients, such as ashwagandha, Rhodiola rosea, ranax ginseng, and holy basil, which contribute to stress resistance, physical endurance, energy metabolism, and the reduction of oxidative stress. Herbal extracts such as turmeric, green tea extract, milk thistle, and ginger root extract may further amplify the anti-inflammatory and antioxidant properties of the formulation 12 while supporting liver health and gut function.

Bioactive polyphenols, such as resveratrol, quercetin, and pterostilbene, also could be included in the formulation 12 to enhance cellular protection and metabolic regulation. The formulation 12 also could include mitochondrial enhancers, such as Coenzyme Q10, alpha-lipoic acid, and carnitine, to bolster mitochondrial energy production, and fatty acid oxidation. The formulation 12 also could include anti-inflammatory agents, such as omega-3 fatty acids, boswellic acids, and cannabidiol, to reduce systemic inflammation and to support brain health. The formulation 12 also could include neuroprotective compounds, such as bacopa monnieri, lion's mane mushroom, and Ginkgo biloba, to provide cognitive resilience and mitigate neurodegenerative risks.

The formulation 12 also could include additional metabolic support agents, such as berberine, chromium, fenugreek, and inositol, to regulate glucose metabolism, enhance insulin sensitivity, and improve hormonal balance. The formulation 12 also could include essential vitamins and minerals, such as B-vitamins, vitamin D, magnesium, selenium, zinc, and iodine, to contribute to energy production, immune function, and enzymatic reactions, ensuring comprehensive systemic support. The formulation 12 also could include botanicals, such as schisandra, astragalus, and neem, as well as emerging compounds like nicotinamide riboside, to extend the benefits of the formulation 12 by supporting NAD+ levels, autophagy, and cellular health. The inclusion of these diverse compounds in the formulation 12 could create a robust and multi-faceted therapeutic strategy, targeting a wide spectrum of cellular and systemic health pathways to optimize vitality, longevity, and resilience against age-related decline.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In use, the multi-ingredient natural products composition 10 enables a method 24 of treating a subject with the urolithin A 14 and the spermidine 16 or a biologically active analog thereof, to improve a biological process in the subject. The method 24 entails provision of the urolithin A 14, the spermidine 16 or a biologically active analog thereof, and one or more of the liquid 18, the excipient 20, and the device 22 configured to render the urolithin A 14 and the spermidine 16 suitable for therapeutic delivery to the subject. Upon producing the formulation 12, it can be delivered to the subject to improve the biological process.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Therefore, the foregoing is considered as illustrative only of the principles of the disclosure. Further, since numerous modifications and changes will readily occur to those skilled in the art, it is not desired to limit the disclosure to the exact combinations, methods, and operations shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A multi-ingredient natural products composition comprising:
a formulation comprising:
urolithin A;
spermidine, or a biologically active analog thereof; and
one or more of a liquid, an excipient, and a device configured for rendering the mixture of natural products suitable for therapeutic delivery to a subject; and
wherein the formulation is configured for improving a biological process in the subject.

2. The multi-ingredient natural products composition of claim 1, wherein:
the urolithin A in the formulation is from 200 mg to 1500 mg; and
the spermidine, or the biologically active analog thereof, in the formulation is from 0.5 mg to 20.0 mg.

3. The multi-ingredient natural products composition of claim 1, wherein:
the urolithin A in the formulation is from 250 mg to 1000 mg; and
the spermidine, or the biologically active analog thereof, in the formulation is from 1.0 mg to 10.0 mg.

4. The multi-ingredient natural products composition of claim 1, wherein the weight ratio of the urolithin A to the spermidine, or the biologically active analog thereof, in the formulation has a range of from 25:1 to 1000:1.

5. The multi-ingredient natural products composition of claim 1, wherein the one or more of the liquid, the excipient, and the device renders the formulation deliverable to the subject as a solution, emulsion, or a suspension by oral delivery, parenteral delivery, topical delivery, or inhalational delivery.

6. The multi-ingredient natural products composition of claim 1, wherein the one or more of the liquid, the excipient, and the device renders the formulation deliverable to the subject orally as a pill, a capsule, a time-release capsule, or a tablet.

7. The multi-ingredient natural products composition of claim 1, wherein the one or more of the liquid, the excipient, and the device renders the formulation deliverable transdermally to the subject.

8. A method of treating a subject with urolithin A and spermidine, or a biologically active analog thereof, to improve a biological process in the subject, the method comprising:
providing the urolithin A, the spermidine, or the biologically active analog thereof;
providing one or more of a liquid, an excipient, and a device configured to render the urolithin A, the spermidine, or the biologically active analog thereof, suitable for therapeutic delivery to the subject;
combining the urolithin A, the spermidine, or the biologically active analog thereof and the one or more of the liquid, the excipient, and the device to generate a formulation; and
delivering the formulation to the subject to improve the biological process.

9. The method of claim 8, wherein:
the urolithin A in the formulation is from 200 mg to 1500 mg; and
the spermidine, or the biologically active analog thereof, in the formulation is from 0.5 mg to 20.0 mg.

10. The method of claim 8, wherein the weight ratio of the urolithin A to the spermidine, or the biologically active analog thereof, in the formulation has a range of from 25:1 to 1000:1.
